(19) United States
(12) Patent Application Publication
(10) Pub. No.: US 2011/0104101 A1
(43) Pub. Date: May 5, 2011

(54) Immunotherapy for Unresectable Pancreatic Cancer

(75) Inventor: Edmund C. Lattime, Piscataway, NJ (US)

(73) Assignee: UNIVERSITY OF MEDICINE AND DENTISTRY OF NEW JERSEY, Somerset, NJ (US)

(21) Appl. No.: 12/920,803

(22) PCT Filed: Mar. 9, 2009
(86) PCT No.: PCT/IB09/00466

§ 371 Date: Dec. 13, 2010

Related U.S. Application Data
(60) Provisional application No. 61/068,301, filed on Mar. 6, 2008.

(51) Int. Cl. A61K 38/19; A61P 35/00; A61K 39/285

(52) U.S. Cl. 424/85.1; 424/199.1

(57) ABSTRACT

The present invention provides a novel cancer immunotherapy comprising a vaccination schedule of both intratumoral and systemic injections followed by peripheral boost injection. The immunotherapy can then be followed by other standard treatment as is known in the art for locoregional or metastatic pancreatic cancer. The present invention further provides a kit for administering the cancer immunotherapy described herein. The present invention further provides a method of decreasing the dose of cancer immunotherapy vaccines.

### CLAIM OF PRIORITY

This application claims priority to U.S. Application No. 61/068,301, filed on Mar. 6, 2008, the disclosure of which is hereby incorporated into this application in their entirety

### BACKGROUND OF THE INVENTION

Pancreatic cancer remains one of the most lethal of malignant solid tumors and the fourth leading cause of death in the United States. It is usually diagnosed in an advanced stage. The American Cancer Society estimated for 2007 that approximately 37,170 Americans will be diagnosed with cancer of the pancreas and 33,370 will succumb to pancreatic cancer; for 2008 the estimates were 33,680 and 34,290, respectively. Only approximately 20% of patients will be considered to have resectable disease and 80% of those will recur after surgery. About 24% of patients with cancer of the pancreas will be alive one year after their diagnosis; only about 5% will live 5 years after diagnosis.

For resected pancreatic cancer, 80-90 percent of tumors are located in the head. Pancreatic carcinoma metastasizes to regional lymph nodes. Perineural, vascular and lymphatic invasion is also frequently seen with in resected specimens. Patients who undergo resection for non-metastatic disease have a 5-year survival of 7-25 percent with a median survival of 11-20 months. The majority of patients develop disease recurrence within two years in sites including commonly retroperitoneum, peritoneum, liver and, less commonly, lung.

Adjuvant chemotherapy or chemoradiotherapy is utilized post operatively, with some controversy as to benefit. Older US data suggests that 5FU+radiation therapy improves survival from 11 to 20 months

A more recent EORTC study, using 5FU and split course radiation, suggests modest benefit with, improving survival from the addition of chemoradiotherapy from a median of 19 to 24 months

A highly criticized trial from a European group suggests benefit from chemotherapy but not concurrent chemoradiotherapy. In a 2×2 design, 289 patients received either observation only, chemotherapy, radiotherapy or the combination. The median survival was 16.9 months among the 69 patients randomly assigned to observation, 13.9 months among the 73 patients randomly assigned to chemoradiotherapy, 19.9 months among the 72 patients randomly assigned to chemoradiotherapy plus chemotherapy and 21.6 months among the 75 patients randomly assigned to chemotherapy, alone.

Patients with unresectable locally advanced, non-metastatic disease have a median survival of 6-11 months. The current treatment for patients with locoregional non-resectable disease is chemotherapy with or without radiation. Support for chemoradiation comes from two early randomized studies. In both randomized trials, the combined modality group had a better survival compared with radiation therapy alone. Nevertheless, a third randomized trial in which 5-FU-based chemotherapy was compared with combined modality chemoradiotherapy, no statistically significant improvement in median survivals was appreciated. No more recent randomized trials are available An ECOG study comparing gemcitabine to gemcitabine 5FU/radiotherapy has apparently closed short of accrual goals.

In advanced and metastatic pancreatic cancer, gemcitabine (at a dose of 1000 mg/m² weekly for 7 consecutive wk followed by a week of rest for the first cycle and then weekly for 3 consecutive wk followed by a week of rest in subsequent cycles) conferred a survival advantage relative to 5-FU. The one year survival with gemcitabine was 18% compared to 3% for 5FU treatment. Other chemotherapy agents have been combined with gemcitabine and in general improve response frequency but without changing overall survival, which remains approximately 6-7 months in large studies. ECOG is currently comparing standard gemcitabine, to fixed-dose rate gemcitabine to gemcitabine+oxaliplatin (GEMOX) in 800 patients, to assess if either of the latter two regimens improves outcome compared to gemcitabine alone. Recently, the combination of erlotinib and gemcitabine was compared to gemcitabine alone. Overall survival was improved only by approximately two weeks with the combination, though one-year survival improved from 17% to 24% with the combination.

Thus, while surgery yields the most favorable outcomes, its role is limited to 20% of the patients and among these patients, less than 25% are likely 5-year survivors. For patients with locoregional disease, current therapy yields a median survival of 20 months and for those with metastatic disease, median survivals of 6-8 months are expected with a one-year survival of approximately 18%-with standard gemcitabine therapy. Clearly better therapies need be identified.

### SUMMARY OF THE INVENTION

The instant invention relates to a novel immunotherapy comprising a vaccination schedule of both intratumoral and systemic injections followed by peripheral boost injection. The immunotherapy can then be followed by other standard treatment as is known in the art for locoregional or metastatic pancreatic cancer.

Certain embodiments of the invention are designed to administer combined intratumoral (PANVAC-F (fowlpox)) and systemic (PANVAC-V (vaccinia)) priming and two peripheral boost injections (PANVAC-F (fowlpox)) over a period of one month prior to the initiation of other standard treatment for locoregional or metastatic pancreatic cancer.

The instant invention also relates to a method of administering cancer immunotherapy comprising administering a vaccine by intratumoral injection; administering a vaccine by systemic injection; and administering a vaccine by peripheral boost injection. In some embodiments, the vaccine administered by intratumoral injection is a replication deficient recombinant fowlpox virus vector vaccine; the vaccine administered by systemic injection is a replication competent recombinant vaccinia virus vector vaccine, and the vaccine administered by peripheral boost injection is a replication deficient recombinant fowlpox virus vector vaccine. In further embodiments, the vaccine administered by intratumoral injection is a replication deficient recombinant fowlpox virus vector vaccine comprising at least one gene coding for a molecule selected from the group consisting of CEA, MUC-1, B7.1, ICAM-1, and LFA-3; the vaccine administered by systemic injection is a replication competent recombinant vaccinia virus vector vaccine comprising at least one gene coding for a molecule selected from the group consisting of CEA, MUC-1, B7.1, ICAM-1, and LFA-3; and the vaccine administered by peripheral boost injection is a replication deficient recombinant fowlpox virus vector vaccine comprising at least one gene coding for a molecule selected from the group consisting of CEA, MUC-1, B7.1, ICAM-1, and LFA-3. In other embodiments, the vaccine administered by intratumoral injection is a replication deficient recombinant fowlpox virus vector vaccine containing genes for CEA, MUC-1, B7.1, ICAM-I, and LFA-3; the vaccine administered by systemic injection is a replication competent recombinant vaccinia virus vector vaccine containing genes for CEA, MUC-1, B7.1, ICAM-I, and LFA-3; and the vaccine administered by peripheral boost injection is a replication deficient recombinant fowlpox virus vector vaccine containing genes for CEA, MUC-1 B7.1, ICAM-I, and LFA-3. Still other embodiments further comprise the administration of rH-GM-CSF.

The invention also relates to a method of administering cancer immunotherapy comprising administering at least one intratumoral injection comprising a replication deficient recombinant fowlpox virus vector vaccine containing genes for CEA, MUC-1, B7.1, ICAM-1, and LFA-3; administering at least one systemic injection comprising a replication competent recombinant vaccinia virus vector vaccine containing genes for CEA, MUC-1, B7.1, ICAM-1, and LFA-3; and administering at least one peripheral boost injection comprising a replication deficient recombinant fowlpox virus vector vaccine containing genes for CEA, MUC-1, B7.1, ICAM-1, and LFA-3. Certain embodiments may further comprise administering at least one injection of rH-GM-CSF.

The present invention relates to a method of administering cancer immunotherapy comprising injecting a patient with a first intratumoral injection of a replication deficient recombinant fowlpox virus vector vaccine containing genes for CEA, MUC-1, B7.1, ICAM-1, and LFA-3; injecting the patient with a parenteral injection of a replication competent recombinant vaccinia virus vector vaccine containing genes for CEA, MUC-1, B7.1, ICAM-1, and LFA-3, and injecting the patient with rH-GM-CSF at the local region of the parenteral injection site immediately thereafter; injecting the patient with a second intratumoral injection of a replication deficient recombinant fowlpox virus vector vaccine containing genes for CEA, MUC-1, B7.1, ICAM-1, and LFA-3; injecting the patient with a first parenteral injection of a replication deficient recombinant fowlpox virus vector vaccine containing genes for CEA, MUC-1, B7.1, ICAM-1, and LFA-3, and injecting the patient with rH-GM-CSF at the local region of the parenteral injection site immediately thereafter; and injecting the patient with a second parenteral injection of a replication deficient recombinant fowlpox virus vector vaccine containing genes for CEA, MUC-1, B7.1, ICAM-1, and LFA-3, and injecting the patient with rH-GM-CSF at the local region of the parenteral injection site immediately thereafter. In some embodiments, the injections of rH-GM-CSF are administered within from 1 to 25 mm of the parenteral injection site. In some embodiments, the steps are performed in the sequence set forth above. In other embodiments, the first two steps are performed on the same day. Other embodiments further comprise injecting the patient with at least one injection of rH-GM-CSF.

In some embodiments of the present invention, the patient may be concurrently undergoing treatment with gemcitabine, 5FU, or a combination thereof.

In other embodiments of the present invention, at least one of the intratumoral injections of a replication deficient recombinant fowlpox virus vector vaccine comprises a dose selected from the group consisting of 1×10⁷ pfu, 1×10⁸ pfu, and 1×10⁹ pfu.

In further embodiments of the present invention, the parenteral injection of a replication competent recombinant vaccinia virus vector vaccine comprises a dose of 2×10⁸ pfu.

In other embodiments of the present invention, at least one of the parenteral injections of a replication deficient recombinant fowlpox virus vector vaccine comprises a dose selected from the group consisting of 1×10⁷ pfu, 1×10⁸ pfu, and 1×10⁹ pfu.

In some embodiments of the present invention, at least one injection of rH-GM-CSF comprises a dose of from 1 to 1000 mcg.

In certain embodiments of the present invention, the gene for CEA contains a single amino acid substitution in one 9-mer, HLA-A2-restricted, immunodominant epitope, wherein said amino acid substitution comprises the substitution of aspartic acid for asparagine at amino acid position 609. In further embodiments of the present invention, the gene for MUC-1 contains a single amino acid substitution in one 10-mer, HLA-A2-restricted, immunodominant epitope, wherein said amino acid substitution comprises the substitution of leucine for threonine at amino acid position 93.

In certain embodiments, the injections may be administered over a period of from 1 to 60 days.

The present invention relates to a kit for the administration of cancer immunotherapy comprising at least one dose of a replication deficient recombinant fowlpox virus vector vaccine containing genes for CEA, MUC-1, B7.1, ICAM-1, and LFA-3 and at least one does of a replication competent recombinant vaccinia virus vector vaccine containing genes for CEA, MUC-1, B7.1, ICAM-1, and LFA-3. In some embodiments, at least one does of a peripheral booster may be included. In further embodiments, at least one dose of rH-GM-CSF may be included. The kit of the present invention may further include instructiosn for the use thereof. In some embodiments, the instructions are in paper or electronic form.

The present invention relates to a method of decreasing the dose of an immunotherapy vaccine comprising administering at least one intratumoral injection of tumor-antigen encoding poxvirus vaccine. In some embodiments, the vaccine is a replication deficient recombinant fowlpox virus vector vaccine. In other embodiments, the vaccine is a replication deficient recombinant fowlpox virus vector vaccine comprising at least one gene coding for a molecule selected from the group consisting of CEA, MUC-1, B7.1, ICAM-1, and LFA-3. In further embodiments, the vaccine is a replication deficient recombinant fowlpox virus vector vaccine containing genes for CEA, MUC-1, B7.1, ICAM-1, and LFA-3. In other embodiments, the vaccine is a replication competent recombinant vaccinia virus vector. In other embodiments, the vaccine is a replication competent recombinant vaccinia virus vector vaccine comprising at least one gene coding for a molecule selected from the group consisting of CEA, MUC-1, B7.1, ICAM-I, and LFA-3. In further embodiments, the vaccine is a replication competent recombinant vaccinia virus vector vaccine containing genes for CEA, MUC-1, B7.1, ICAM-I, and LFA-3.

### DETAILED DESCRIPTION OF THE INVENTION

Research conducted in connection with the instant invention was the first to suggest that the intratumoral injection of tumor-antigen encoding poxvirus vectors resulted in the generation of a stronger antitumor immune response than when the same vectors were administered subcutaneously in preclinical models of bladder cancer and breast cancer. It was found, in a mouse bladder cancer model, that tumor-bearing mice manifested systemic tolerance to the tumor antigen and an inability to respond systemically to vaccine. It was demonstrated, using state of the art identification of tumor-antigen specific CD8 T cells using tetramers, a surprising accumulation or expansion of tumor specific cells in the tumor draining lymph node. Tumor bearing mice were immunized intratumorally with recombinant vaccinia encoding tumor antigen as described herein. The result of this intratumoral immunization was that the anergic mice became systemically immune to the tumor antigen. In the case of the breast cancer model, this immunization resulted in a rejection of tumor in a number of mice. These findings serve as the basis for the strategy of immunizing to the tumor antigen intratumorally, as further described herein.

It has been proposed that the proper engagement of the T cell receptor and costimulatory receptor requires the expression of both antigen and costimulatory molecules, respectively, in the same cell. Therefore, co-expression of costimulatory molecules and antigens using a single recombinant vector or an admixture of two vectors presents the potential of cooperation among these proteins to enhance T cell activation. A number of preclinical studies have supported the feasibility of this approach. Immunization of mice with admixtures of two recombinant vaccinia viruses, one expressing B7.1 (rV-B7.1) and the other expressing CEA (rV-CEA), resulted in increased CEA-specific immune responses and enhanced protection against challenge with CEA-bearing tumors as compared to immunization with rV-CEA alone. Co-expression of CEA and B7.1 in a single recombinant vaccinia virus was also more effective than the admixture of rV-CEA and rV-B7.1 with respect to eliciting CEA-specific immunity. Similar enhancement of antitumor immunity was observed in murine studies using an admixture of rV-MUC-1 and rV-B7.1.

Recombinant vectors co-expressing the three TRICOM costimulatory molecules have been shown to have synergistic effects on antitumor responses as compared to vectors expressing individual costimulatory molecules. For example, T cell proliferation and antitumor immunity using recombinant vaccinia virus co-expressing murine TRICOM were much greater than the sum of responses seen using vaccinia virus expressing individual costimulatory molecules. In addition, mice immunized with a recombinant vaccinia virus co-expressing CEA and murine TRICOM exhibited greater immune responses and antitumor responses than mice immunized with a recombinant vaccinia virus co-expressing CEA and murine B7.1. Enhanced antitumor immunity was also observed in mice that were transgenic for CEA. Therefore, PANVAC-V (vaccinia) and PANVAC-F (fowlpox) have been designed to simultaneously express CEA and MUC-1 together with B7.1, LFA-3, and ICAM-1.

Maximal immune responses are achieved when two different pox virus vectors—vaccinia virus and fowlpox virus—are used in combination in prime-boost regimens. Host immune responses to vaccinia restrict its replication and thus limit its ability to continue to elicit tumor-specific immune responses after multiple vaccinations. Consequently, vaccinia-based vaccines can be used to immunize an individual only a limited number of times. Productive fowlpox virus infection is restricted in vivo to avian species and in vitro to cells derived from avian species. A number of studies have shown that immunization of mammalian species by recombinant fowlpox virus can stimulate both humoral and cell-mediated immunity to the expressed transgene.

Vaccinia virus has been used for over 200 years as a vaccine for smallpox and has a well-established safety profile. The virus actively replicates in human cells, resulting in the presentation of high levels of antigen to the immune system over a period of one to two weeks, substantially increasing the potential for immune stimulation. The immune response specific to vaccinia then eliminates the virus. As a result of its safety profile and ability to elicit both humoral and cell-mediated immunity in humans, the vaccinia virus (genus Orthopoxvirus) was chosen as one of the vectors to deliver MUC-1, CEA, and TRICOM.

Fowlpox virus, like vaccinia, is a member of the Poxviridae family (genus Avipoxvirus) that can infect mammalian cells and express inserted transgenes to stimulate both humoral and cellular immunity. Fowlpox cannot replicate in non-avian species, making systemic infections unlikely and making it potentially safer than a replicative virus. Results from NCI-sponsored Phase I and II studies of other fowlpox-based vaccines support the safety of this vector.

Recombinant pox viruses can infect antigen-presenting cells, including dendritic cells and macrophages, resulting in efficient expression of tumor associated antigens (TAAs) simultaneously with costimulatory molecules required for the elicitation of T cell responses. TAAs expressed by recombinant pox viruses are presented to the immune system together with highly immunogenic virus proteins, which may act as adjuvants to enhance immune responses to the TAAs. Thus, the use of recombinant pox virus vectors for the presentation of TAAs to the immune system results in the generation of killer T cells that specifically destroy the selected tumor with little incremental toxicity.

The immune responses to vaccinia do not inhibit fowlpox virus, which can be given numerous times. Therefore, by priming with recombinant vaccinia virus and then boosting repeatedly with the corresponding recombinant fowlpox virus, maximum immune responses to the expressed tumor antigens can be obtained. This phenomenon has been demonstrated in animal models and has been supported by results of ongoing clinical trials.

GM-CSF has been shown to be an effective vaccine adjuvant because it enhances antigen processing and presentation by dendritic cells. Experimental and clinical studies suggest that recombinant GM-CSF can boost host immunity directed at a variety of immunogens.

Using murine tumor models, several researchers have now shown that modification of tumor cells to enhance GM-CSF expression, using retroviral vectors or vaccinia virus vectors, results in enhanced tumor-specific immune responses capable of effecting tumor destruction. Furthermore, this immune response is effective against not only the engineered, GM-CSF-expressing tumors, but also against unaltered tumor cells. An embodiment of the present invention uses GM-CSF locally, at the vaccination site, to enhance immune responses elicited by the recombinant vaccines.

Preclinical and/or clinical data indicate that the prime-boost approach with the GM-CSF adjuvant merits application as an antitumor treatment for the following reasons:
Presentation of TAAs by recombinant vaccinia or fowlpox viruses results in antigen-specific immune responses. The modified epitopes in CEA and MUC-1 may elicit an enhanced immune response in patients who express the HLA-A2 genotype.
Antitumor activity is enhanced when both antigens and costimulatory molecules are presented to the host.
Priming with a recombinant vaccinia virus prior to administering a series of recombinant fowlpox virus inoculations has been shown to greatly enhance immune responses to the target antigen.
GM-CSF is a potent vaccine adjuvant capable of augmenting the immune response.

The biological agents PANVAC-V (vaccinia) and PANVAC-F (fowlpox) are recombinant vaccinia and fowlpox viruses, respectively, encoding the genes for MUC-1, CEA, and three human costimulatory molecules, B7.1, ICAM-1, and LFA-3. Human rH-GM-CSF will be administered at the vaccination site on the day of each vaccination and for 3 days thereafter.

PANVACT™-V is a replication competent recombinant vaccinia virus vector vaccine containing genes for human CEA, MUC-1 and three co-stimulatory molecules (designated TRICOM™): B7.1, ICAM-1 (intercellular adhesion molecule-1), and LFA-3 (leukocyte function-associated antigen-3). The CEA gene coding sequence is modified to code for a single amino acid substitution (aspartic acid, instead of asparagine at amino acid position 609) in one 9-mer, HLA-A2-restricted, immunodominant epitope designed to enhance immunogenicity. The MUC-1 gene coding sequence is also modified to code for a single amino substitution (leucine, instead of threonine at amino acid position 93) in one 10-mer, HLA-A2-restricted, immunodominant epitope designed to enhance immunogenicity.

PANVACT™-F is a replication deficient recombinant fowlpox virus vector vaccine containing the same recombinant gene combination. These recombinant virus vectors have been generated as the result of a large series of preclinical and clinical studies testing the individual gene products alone and in combination.

At a high level, certain embodiments of the instant invention utilize a five-component strategy for generating an improved immune response: 1) altering the amino acid sequence of the tumor antigen to enhance its immunogenicity; 2) utilizing T-cell co-stimulatory molecules to enhance the T-cell response; 3) utilizing a viral vector to enhance presentation; 4) using two different types of vaccine for the primer and boost vaccine; and 5) using rH-GM-CSF to enhance recruitment of dendritic cells.

Virtually all pancreatic and periampullary cancers express CEA and most produce MUC 1. Carcinoembryonic antigen (CEA) is an 180,000 dalton glycoprotein that is over-expressed on most adenocarcinomas of the colon, rectum, stomach, and pancreas, as well as on breast cancers and non-small-cell lung cancers. The immunogenicity of CEA in humans has been demonstrated in several clinical trials. The development of humoral and T cell immunity to CEA as a result of immunization with a CEA anti-idiotype vaccine has been previously reported. In addition, a number of clinical trials using recombinant vaccinia and/or avipox viruses expressing CEA have been conducted. These trials demonstrated for the first time that CEA, when expressed by a recombinant pox virus, can elicit or enhance human immune responses capable of recognizing and destroying tumor cells that express CEA.

Protein antigens are presented to cytotoxic T lymphocytes as small peptides (approximately 9-10 amino acids long) bound to class I molecules of the major histocompatibility (MHC) complex. One strategy to increase the immunogenicity of a self-antigen such as CEA is to modify selected epitopes within the protein sequence to enhance their binding to MHC class I alleles or to the T cell receptor. One such modified epitope, designated CAP-1(6D), was shown to be 100-1000 times more efficient than the native CAP-1 peptide in the induction of CAP-1-specific cytotoxic T lymphocytes (CTLs). In contrast to the native peptide, CAP-1(6D) was able to induce CD8+ CTLs from normal peripheral blood mononuclear cells that were able to recognize both the modified and native peptides. In addition, these CTLs recognized and lysed tumor cell lines expressing CEA. These studies indicate that CEA glycoprotein containing the modified peptide may be more efficient in and capable of eliciting and sustaining antitumor responses than unmodified glycoprotein.

Mucin-1 (MUC-1) is a glycosylated transmembrane protein that is uniquely characterized by an extracellular domain that consists of a variable number of tandem repeats of 20 amino acids. Pancreatic adenocarcinomas aberrantly glycosylate as well as overexpress MUC-1. Immunization with a MUC-1 peptide or a recombinant vaccinia virus expressing MUC-1 has been shown to induce MUC-1-specific immune responses in pancreatic and breast cancer patients. Thus, immunization of pancreatic cancer patients with pox viruses expressing MUC-1 may boost the antitumor immunity against their cancers.

As described above for CEA, a selected epitope within the MUC-1 protein sequence was modified to increase its binding to the MHC class I A2 allele in order to enhance the immunogenicity of the polypeptide. This epitope, designated P93L, was shown to be more efficient than the native P92 peptide in the stimulation of gamma-interferon production by MUC-1-specific T cell lines. P93L was also able to induce CD8+ CTLs from peripheral blood mononuclear cells collected from pancreatic patients that could recognize and lyse tumor cell lines expressing native MUC-1. These studies indicate that MUC-1 glycoprotein containing the modified peptide may be more efficient in and capable of eliciting and sustaining antitumor responses than unmodified glycoprotein. In addition, the number of tandem repeats in the native MUC-1 gene varies in humans, with a range of 21 to 125 copies per gene. A recombinant vaccinia virus, rV-MUC-1, was generated using a MUC-1 gene that contains the signal sequence, six copies of the tandem repeat sequence, and the 3′ unique coding sequence. Preclinical studies in a murine tumor model system demonstrated that vaccination with this recombinant pox virus expressing MUC-1 caused regression of MUC-1-bearing tumors.

At least two signals are required for activation of naive T cells by antigen-presenting cells (APCs): (1) an antigen-specific signal, delivered through the T cell receptor by an antigen presented in the context of a MHC molecule and (2) an antigen-independent or costimulatory signal, which is needed for cytokine production and T cell proliferation.

At least three distinct molecules normally found on the surface of “professional APCs” can provide this second costimulatory signal: B7.1, intracellular adhesion molecule-1 (ICAM-1), and leukocyte function-associated antigen-3 (LFA-3). These molecules function through non-redundant signaling pathways. B7.1 is the ligand for the T cell surface receptor CD28 and delivers a stimulatory signal when bound to CD28. ICAM-1 binds to its ligand LFA-1, which is expressed on the surface of lymphocytes and granulocytes. LFA-3, a member of the immunoglobulin gene superfamily, binds to CD2, found on thymocytes, T cells, B cells, and natural killer cells.

The combination of B7.1, LFA-3, and ICAM-1 has been designated as “TRICOM”, for TRIad of COstimulatory Molecules. Recombinant vectors that simultaneously express TRICOM together with a tumor-associated antigen elicit significantly higher immune responses and confer enhanced protection against challenge with tumors expressing the corresponding antigen. Such antitumor responses can be elicited even when the target tumor-associated antigen represents a “self” antigen. For example, in tumor immunotherapy studies using transgenic mice that expressed the human tumor antigen carcinoembryonic antigen (CEA), animals with established CEA-positive hepatic carcinoma metastases were administered weekly vaccinations for four weeks with a vaccinia recombinant that expressed CEA and TRICOM; murine GM-CSF and IL-2 were also administered to further enhance vaccine-specific immune responses. Of the sixteen treated mice, nine (56%) remained alive through 25 weeks. By contrast, in the control group (which received non-recombinant vaccinia plus cytokines), only one of nineteen (5%) survived past 16 weeks.

Over 700 cancer patients, most with metastatic disease, have been treated to date with pox virus-based vaccines in CTEP-sponsored clinical trials, including over 100 patients who received recombinant human GM-CSF in combination with the vaccines. Although the reported data were collected from multiple clinical trials which differed in dose, route of administration, dosage regimen, use of combination therapy, as well as type and stage of cancer, overall these studies have demonstrated: (i) the safety and tolerability profile of pox virus-based vaccines in completed and ongoing clinical studies in cancer patients; (ii) clinically relevant immunologic responses, particularly cytotoxic T cell responses, directed against the tumor-associated antigen expressed by the vaccines, obtained in a significant number of patients after vaccination; (iii) evidence suggesting that generation of such immune responses is accompanied by clinical benefit, such as increased survival in pilot studies of patients with CEA-bearing tumors; and (iv) clinically unexpected, objective responses anecdotally noted in several patients with advanced pancreatic cancer who have received pox virus-based vaccines expressing CEA. The above-referenced studies are described in the following publications, which are hereby incorporated by reference in their entireties: Investigator's brochure for PANVAC-VF Version 3.1.2005; Bohle, A. and Brandau, S. Immune Mechanisms in bacillus Calmett-Guerin immunotherapy for superficial bladder cancer, J Urol, 170: 964-969, 2003; Mastrangelo, M. J. et al., Intratumoral recombinant GM-CSF-encoding virus as gene therapy in patients with cutaneous melanoma, Cancer Gene Ther, 6: 409-422, 1999; Dipaola, R. et al., Phase I Trial of Pox PSA vaccines (PROSTVAC(R)-VF) WITH B7-1, ICAM-1, AND LFA-3 co-stimulatory molecules (TRICOM trademark) in Patients with Prostate Cancer, J Transl Med, 4: 1, 2006; Marshall, J. L. et al., Phase I study of sequential vaccinations with fowlpox-CEA(6D)-TRICOM alone and sequentially with vaccinia-CEA(6D)-TRICOM, with and without granulocyte-macrophage colony-stimulating factor, in patients with carcinoembryonic antigen-expressing carcinomas, J Clin Oncol, 23: 720-731, 2005.

### EXAMPLES

### Example 1

### Preliminary Studies with Vaccinia

In-vivo murine bladder cancer studies: To determine if recombinant vaccinia infects/transfects tumor, and perhaps normal mucosa, in-vivo, the influenza hemagglutinin and nuclear protein encoding vaccinia recombinants (10⁷ PFU) were instilled via urethral catheters into the bladders of C57BL/6 mice bearing the MB49 tumor, and following 8 hrs. the mice were euthanized, their bladders removed, sectioned and stained for the two antigens immunohistochemically. When stained for the nuclear protein, substantial infection/transfection of the growing MB49 tumor was found. While infected/transfected normal mucosa also stained for the encoded antigen, there was an apparent preferential staining in tumor. Whether this was due to a lack of protective glycosaminoglycan layer on the tumor or was peculiar to early studies, what was clear is that a high efficiency of infection/transfection of bladder tumor cells was obtained following intravesical instillation of the virus. There was seemingly no acute toxicity.

While it has been shown that recombinant vaccinia infected bladder tumors in-vitro and in-vivo in naive animals, it was important to demonstrate infection/transfection in mice immune to the virus which would be analogous to patients who had been vaccinated or following the first treatment. Intravesical administration of VAC-NP (reporter gene construct) infects/transfects intravesically growing MB49 in the presence of systemic immunity. Mice were injected i.p. with native vaccinia (shown to result in systemic anti-vaccinia immunity based on cytotoxic T lymphocyte (CTL) generation), the mice instilled with MB49 tumor intravesically, and 2 weeks later when tumor had been established, were instilled with VAC-NP. Twelve hours later the bladders were removed and stained immunohistochemically for the recombinant NP antigen which was positive. In addition, cytologic changes including ballooning degeneration and intranuclear inclusion bodies were noted. Thus, systemic immunity to vaccinia which would be expected to be present in adult patients and following initial vaccinia treatments does not prevent intravesical tumor infection/transfection.

To demonstrate that VAC was able to recruit lymphocytes to the bladder wall and generate a systemic immune response, graded numbers of VAC from 10 to 10⁶ were instilled into bladders of C57BL/6 mice. Following 2 weeks incubation, spleen cells were removed from the mice, restimulated in vitro with VAC for 7 days and the resultant cells tested for their ability to lyse the VAC-infected MB49 tumor target. As few as 10 PFU instilled intravesically resulted in significant VAC immunity demonstrating its high degree of immunogenicity.

Human melanoma studies: As a prelude to studying the effects of intralesional recombinant vaccinia in human melanoma, a feasibility study using intralesional wild-type vaccinia (BB-IND 5002) was conducted.

The conclusions were as follows: (1) Despite historical and physical evidence of prior vaccination, all patients experienced a major reaction with pustule formation at the initial cutaneous inoculation site. (2) In immunocompetent patients, very large amounts of vaccinia can be administered safely (10⁷ PFU per injection; 12×10⁷ PFU total). (3) It is possible to locally infect tumor cells at virus injection sites (with viral protein production).

Following the wild type vaccinia study, patients with superficial metastatic melanoma have been treated using recombinant vaccinia encoding human GMCSF (BB-IND 6486). To date seven patients have been studied and the results described in detail in reference. In summary, in all but the two patients with the highest tumor burden, injected lesions regressed, with noninjected lesions regressing in 4 of 7. As noted above for the wild type, it has been demonstrated that repeated treatment in the face of maximal titers of anti-vaccinia antibody consistently demonstrated the ability to infect/transfect tumor with the encoded GMCSF gene. Importantly, there was no significant toxicity noted.

Human bladder studies: Phase I study of the safety parameters in the intravesical administration of vaccinia have also been completed. These studies followed the human melanoma experience above, were supported by an amendment to BB-IND-5002, and included patients with invasive bladder tumor prior to cystectomy as is described here using the recombinant fowlpox virus. Vaccinia virus was provided by the CDC from their stocks kept after ending the smallpox vaccination program. Four patients were treated. Immunocompetent patients were vaccinated on the upper arm and following a demonstrated “take” indicating an anti-vaccinia response, escalating doses of vaccinia were instilled intravesically for a total of four doses with the last dose given 24 hrs. prior to cystectomy. In the first patient, doses of 1, 5, and 10×10⁶ were given, in the second 10, 25, and 100×10⁶ and in the third and fourth 25, 100, and 100×10⁶ were administered. Upon examination of the cystectomy specimens, significant vaccinia induced inflammatory infiltrates were seen in the mucosa and submucosa of the patients who received the higher doses (patients 2-4). Post vaccinia mucosa showed virally infected cells with vacuolization. Side effects were limited and consisted only of transient dysuria. Excellent patient tolerance of the intravesical vaccinia and the significant immune infiltrates seen following instillation support the trial described in this application.

### Example 2

### Intra-Pancreatic Injection of ONYX-015, an E1B-55 kDa Gene-Deleted, Replication-Selective Adenovirus

There have been 2 trials of intratumoral ONYX-015 in patients with non-resectable pancreatic cancer. ONYX-015 is a conditionally replicating adenovirus which was developed as a potential oncolytic agent in tumors with abnormalities in p53 tumor suppressor function.

In the first study, a phase I dose escalation study of ONYX-015 in patients with unresectable pancreatic cancer, ONYX-015 was administered via CT-guided injection (n=22 patients) or intraoperative injection (n=1) into pancreatic primary tumors every 4 weeks until tumor progression. Interpatient dose escalation was carried out with at least three patients per dose level from 10⁸ p.f.u. up to the 10″ p.f.u. dose level (two patients treated at this dose). Injection of ONYX-015 into pancreatic carcinomas was well-tolerated. Mild, transient pancreatitis was noted in only one patient. Dose-escalation proceeded to the highest dose level. Neutralizing antibodies were present in all patients. After injection, ONYX-015 was detectable in the blood 15 min later, but not between 1 and 15 days later. Viral replication was not documented, however, in contrast to trials in other tumor types. No objective responses were demonstrated. Intratumoral injection of an E1B-55 kDa region-deleted adenovirus into primary pancreatic tumors was feasible and well-tolerated at doses up to 10¹¹ p.f.u. (2×10¹² particles), but viral replication was not detectable.

In the second study, ONYX-015 was delivered via endoscopic ultrasound guidance as we describe here. Twenty-one patients with locally advanced adenocarcinoma of the pancreas or with metastatic disease, but minimal or absent liver metastases, underwent eight sessions of ONYX-015 delivered by EUS injection into the primary pancreatic tumor over 8 weeks. The final four treatments were given in combination with gemcitabine (i.v., 1,000 mg/m²). Patients received 2×10¹⁰ (n=3) or 2×10¹¹ (n=18) virus particles/treatment. After combination therapy, 2 patients had partial regressions of the injected tumor, 2 had minor responses, 6 had stable disease, and 11 had progressive disease or had to go off study because of treatment toxicity. No clinical pancreatitis occurred despite mild, transient elevations in lipase in a minority of patients. Two patients had sepsis before the institution of prophylactic oral antibiotics. Two patients had duodenal perforations from the rigid endoscope tip. No perforations occurred after the protocol was changed to transgastic injections only. This study indicated that ONYX-015 injection via EUS into pancreatic carcinomas by the transgastic route with prophylactic antibiotics is feasible and generally well tolerated either alone or in combination with gemcitabine. Transgastric EUS-guided injection as we propose here was shown to be a practical and safe method of delivering biological agents to pancreatic tumors.

These studies support the feasibility and safety of injecting recombinant virus into the pancreas.

### Example 3

### Study Utilizing PANVAC-F and PANVAC-V

Patients will be identified as locally unresectable or with only small volume metastatic disease by the gastroenterologist and surgeons and referred for consideration of protocol therapy. Patients must have a histologic or cytologic documentation of adenocarcinoma prior to study entry.

The vaccination schedule is designed to administer combined intratumoral (PANVAC-F (fowlpox)) and systemic (PANVAC-V (vaccinia)) priming and two peripheral boost injections (PANVAC-F (fowlpox)) over a period of one month prior to the initiation of other standard treatment for locoregional or metastatic pancreatic cancer.

Day 1: A patient will be NPO for eight hours prior to injection. EUS with injection of PANVAC-F (fowlpox) intratumorally will be done. Prior to injection of the PANVAC-F (fowlpox), patients will undergo pancreas fine needle aspiration (FNA) and core biopsy. Patients will then be injected intratumorally with PANVAC-F (fowlpox) at the indicated dose in a volume of 0.5 cc.

Day 1-2: The patient will return afternoon of Day 1 or Day 2 (determined by the time of the first injection and other patient logistics) for the first parenteral injection of 2×10⁸ pfu PANVAC-V (vaccinia). Vaccination will be via SC inoculation of the upper outer right deltoid or thigh. Immediately following vaccination, a patient will receive 100 μg rH-GM-CSF SC within 5 mm of the site of vaccination.

Days 2-5: Patients will return to the clinic for the next three days for an additional SC injection of 100 μg of rH-GM-CSF within 5 mm of the site of vaccination. The actual study day on which the 3 consecutive injections of rH-GM-CSF will be given will be determined by the day the 1^{st} S.C. systemic injection is received (Day 1 or Day 2) (for a total of 4 injections). Patients will undergo toxicity assessment and blood work on Day 4.

Day 8:Patients will return for toxicity assessment.

Days 10-12: A CT scan will be obtained to assess for the presence of complications associated with the PANVAC-F injection including severe pancreatitis, abscesses or hemorrhage. If the CT scan does not show evidence of these complications, the patient will be treated with Panvac-F on Day 15/16.

Days 15: The patient will be NPO for eight hours prior to injection. Four CPT tubes (10 ml) and 1 serum tube of blood (10 ml) will be drawn for immune studies. An endoscopy will be done to further assess changes to the pancreas and surrounding lymph nodes and to inject a second dose of PANVAC-F as described for Day 1. Prior to injection of the PANVAC-F (fowlpox), patients will undergo pancreas fine needle aspiration (FNA) and core biopsy Patients will remain under observation with q 1 h vital signs and assessments for pain or discomfort for three hours. Patients will be discharged from the GI suite after eating and tolerating a light meal. The patient will return in the afternoon of the day of or the day following the EUS and vaccine injection (determined by the time in the day of the intrapancreatic injection and other patient logistics) for the first injection of subcutaneous PANVAC-F (fowlpox) (1×10⁹ PFU) given into the opposite upper outer deltoid or thigh from that used for the initial subcutaneous immunization with Panvac-V. Immediately following vaccination, a patient will receive 100 μg rH-GM-CSF SC within 5 mm of the site of vaccination.

Days 16-19 Patients will receive rH-GM-CSF 100 mcg SC within 5 mm of the site of vaccination at home or in the clinic for the subsequent three days. The actual study day on which the 3 consecutive injections of rH-GM-CSF will be given will be determined by the day the 1^{st} S.C. systemic injection is received (Day 15 or Day 16) (a total of 4 GM-CSF injections). Toxicity assessment and blood work will be done on Day 18.

Day 29-32 The patient will return Day 29 (+/−1 day) for the second injection of parenteral PANVAC-F (fowlpox) (1×10⁹ PFU). Four CPT tubes (10 ml) and 1 serum tube of blood (10 ml) will be drawn for immune studies. Vaccination will be via SC inoculation of the alternative upper outer deltoid or thigh. Immediately following vaccination, a patient will receive 100 μg rH-GM-CSF SC within 5 mm of the site of vaccination. Patients can receive rH-GMCSF 100 mcg SC within 5 mm of the site of vaccination at home or in the clinic for the subsequent three days. Patients will be assessed for laboratory or radiographic evidence of tumor response or toxicity.

Day 35: Patients may also initiate treatment with standard of care treatment from the local medical and radiation oncologist as considered appropriate for the disease state. (e.g. radiation +5FU or gemcitabine for locoregional disease or gemcitabine-based therapy, alone, for locoregional or metastatic disease). It is expected that systemic chemotherapy might consist of weekly gemcitabine, using the Burris schedule of seven weeks of weekly treatment for the first eight weeks, followed by three weekly treatments every four weeks. However, specific treatment decisions will be left to the discretion of the treating medical oncologist. Similarly, radiation therapy and chemotherapy will allow for either gemcitabine or 5FU therapy, at the discretion of the treating oncologists. Standard dose modifications for these treatments will apply, as determined by the local oncologist.

Days 43-46: Patients with stable or improving pancreatic cancer, by laboratory assessment, radiographic assessment, or physician assessment and with no irreversible or dose-limiting toxicity may start to receive monthly parenteral PANVAC-F (fowlpox)(1×10⁹ PFU). The patient will return Day 43 (+/−1 day). Four CPT tubes (10 ml) and 1 serum tube of blood (10 ml) will be drawn for immune studies prior to vaccination. Vaccination parenteral PANVAC-F (fowlpox) (1×10⁹ PFU) will be via SC inoculation of the alternate upper outer deltoid or thigh. Immediately following vaccination, a patient will receive 100 μg rH-GM-CSF SC within 5 mm of the site of vaccination followed by as additional three days of rH-GM-CSF. Patients can receive rH-GMCSF 100 mcg SC within 5 mm of the site of vaccination at home or in the clinic for the subsequent three days. Vaccinations will be scheduled for the day for one-two days following gemcitabine chemotherapy to avoid rH-GM-CSF being given at the same time as gemcitabine, Patients receiving continuous infusion 5FU concurrent with radiation therapy, shall stop 5FU for the day of vaccination and for three following days of GMCSF therapy. Radiation therapy can continue. A suggested day for vaccine is Thursday, to minimize the days 5FU-RT combination cannot be given. CBC, assuring granulocyte count ≧1200 cells/mm³, will have been obtained prior to the week's gemcitabine dose.

Patients with progressive cancer, by laboratory or imaging studies, and/or with deteriorating performance status, or who have toxicity from the treatment precluding further therapy will be removed from study and offered alternative treatment with standard of care treatment from the local medical and radiation oncologist as considered appropriate for the disease state. (e.g. radiation +5FU or gemcitabine for locoregional disease or gemcitabine-based therapy, alone, for locoregional or metastatic disease).

Monthly: Patients with no irreversible or dose limiting toxicity will receive the parenteral PANVAC-F (fowlpox) immediately followed by 100 μg rH-GM-CSF within 5 mm of the site of vaccination followed by an additional three days of rH-GM-CSF (GM-CSF will be injected on the day of vaccination and on each of 3 following days for a total of 4 injections). Four CPT tubes (10 ml) and 1 serum tube of blood (10 ml) will be drawn for immune studies prior to vaccination. Vaccine will continue to be administered one-two days following chemotherapy or on a chemotherapy “Off” week. Patients will receive vaccine only if granulocyte count is >1200 cells/mm³. PANVAC-F (fowlpox) plus rH-GM-CSF may continue to be administered monthly in the absence of toxicity or tumor progression. Vaccine will continue to be administered one-two days following chemotherapy or on a chemotherapy “Off” week. Radiation therapy can continue. A suggested day for vaccine is Thursday, to minimize the days 5FU-RT combination cannot be given.

Patients will receive vaccine only if granulocyte count is >1200 cells/mm³ Imaging studies will be assessed for stability, response or progression every two months, following the Day 29-32 scan.

### Dose Escalation Schedule

Only 2 dose levels are anticipated starting at Level 1:

PANVAC-F (fowlpox) Intratumoral Dose Escalation

| Dose Level | Intratumoral Dose of PANVAC-F (fowlpox) |
|---|---|
| Level-1 | 1 × 10⁷ pfu |
| Level 1* | 1 × 10⁸ pfu |
| Level 2 | 1 × 10⁹ pfu |

*Dose Level 1 is the starting dose level
Systemic vaccine doses: PANVAC-V (vaccinia) 2 × 10⁸ pfu subcutaneously
PANVAC-F (fowlpox) 1 × 10⁹ pfu subcutaneously
GM-CSF 100 mcg subcutaneously

### Intratumoral EUS Injection Procedures:

All patients have a biopsy-proven diagnosis of pancreatic cancer prior to study entry. EUS will be performed in the standard fashion to identify the neoplasm, perform loco-regional staging, and perform FNA for diagnostic purposes as indicated by the individual patient's additional diagnostic or staging needs. Two additional tissue samples of the neoplasm will be obtained with a 19 gauge, core-needle in order to ascertain a baseline histological assessment of inflammation and to serve as control tissues for the correlative studied.

### Intratumoral Administration of Vaccine:

The 22 gauge FNA needle has a volume of 0.4 mL. A syringe containing 0.9 mL will be affixed to the 22 gauge FNA needle which will be primed with 0.4 mL (the amount needed to fill the needle). The needle will be advanced through the working channel of the EUS instrument. The tumor will be punctured at its most central location and the needle advanced through the tumor up to the border between tumor and normal tissue. Injection of 0.5 ml PANVAC-F (fowlpox) will then be performed into the tumor while slowly withdrawing the needle backwards, so that the entire volume of vaccine is administered into the tumor under direct EUS visualization. Patients with small tumors in whom significant resistance is encountered during injection will have the vaccine volume delivered in two aliquots by repeating the above maneuver another time in an intratumoral position a few mm away from the initial site. Based on published data (ONYX), up to 10 ml of liquid (or up to 20% of the calculated tumor volume) can be safely injected into pancreatic adenocarcinomas by the technique described above.

1. A method of administering cancer immunotherapy comprising:
a. administering a vaccine by intratumoral injection;
b. administering a vaccine by systemic injection; and
c. administering a vaccine by peripheral boost injection.

2. The method of claim 1, wherein the vaccine administered by peripheral boost injection is the same vaccine as the vaccine administered by intratumoral injection or is the same vaccine administered by systemic injection.

3. (canceled)

4. The method of claim 1, wherein
a. the vaccine administered by intratumoral injection is a replication deficient recombinant fowlpox virus vector vaccine; and
b. the vaccine administered by systemic injection is a replication competent recombinant vaccinia virus vector vaccine.

5. The method of claim 1, wherein
a. the replication deficient recombinant fowlpox virus vector vaccine comprises at least one gene coding for a molecule selected from the group consisting of CEA, MUC-1, B7.1, ICAM-1, and LFA-3; and
b. the replication competent recombinant vaccinia virus vector vaccine comprises at least one gene coding for a molecule selected from the group consisting of CEA, MUC-1, B7.1, ICAM-1, and LFA 3.

6. The method of claim 1, wherein
a. the replication deficient recombinant fowlpox virus vector vaccine contains genes for CEA, MUC-1, B7.1, ICAM-I, and LFA-3; and
the replication competent recombinant vaccinia virus vector vaccine contains genes for CEA, MUC-1, B7.1, ICAM-I, and LFA-3.

7. The method of claim 1, further comprising administering rH-GM-CSF.

8. (canceled)

9. (canceled)

10. A method of administering cancer immunotherapy comprising:
a. injecting a patient with a first intratumoral injection of a replication deficient recombinant fowlpox virus vector vaccine containing genes for CEA, MUC-1, B7.1, ICAM-1, and LFA-3;
b. injecting the patient with a parenteral injection of a replication competent recombinant vaccinia virus vector vaccine containing genes for CEA, MUC-1, B7.1, ICAM-1, and LFA-3, and injecting the patient with rH-GM-CSF at the local region of the parenteral injection site immediately thereafter;
c. injecting the patient with a second intratumoral injection of a replication deficient recombinant fowlpox virus vector vaccine containing genes for CEA, MUC-1, B7.1, ICAM-1, and LFA-3;
d. injecting the patient with a first parenteral injection of a replication deficient recombinant fowlpox virus vector vaccine containing genes for CEA, MUC-1, B7.1, ICAM-1, and LFA-3, and injecting the patient with rH-GM-CSF at the local region of the parenteral injection site immediately thereafter; and
e. injecting the patient with a second parenteral injection of a replication deficient recombinant fowlpox virus vector vaccine containing genes for CEA, MUC-1, B7.1, ICAM-1, and LFA-3, and injecting the patient with rH-GM-CSF at the local region of the parenteral injection site immediately thereafter.

11. The method of claim 10, wherein at least one of the injections of rH-GM-CSF are administered within from 1 to 25 mm of the parenteral injection site.

12. The method of claim 10, wherein the steps are performed in the sequence of a, b, c, d, e.

13. (canceled)

14. (canceled)

15. The method of claim 1, wherein the injections are administered to a patient that is concurrently undergoing treatment with gemcitabine, SFU, or a combination thereof.

16. (canceled)

17. (canceled)

18. (canceled)

19. (canceled)

20. The method of claim 6, wherein the gene for CEA contains a single amino acid substitution in one 9-mer, HLA-A2-restricted, immunodominant epitope, wherein said amino acid substitution comprises the substitution of aspartic acid for asparagine at amino acid position 609.

21. The method of claim 6, wherein the gene for MUC-1 contains a single amino acid substitution in one 10-mer, HLA-A2-restricted, immunodominant epitope, wherein said amino acid substitution comprises the substitution of leucine for threonine at amino acid position 93.

22. The method of claim 1, wherein pancreatic cancer is treated.

23. The method of claim 1, wherein the injections are administered over a period of time of from 1 to 60 days.

24. (canceled)

25. (canceled)

26. A kit for the administration of cancer immunotherapy comprising:
a. at least one dose of a replication deficient recombinant fowlpox virus vector vaccine containing genes for CEA, MUC-1, B7.1, ICAM-1, and LFA-3; and
b. at least one dose of a replication competent recombinant vaccinia virus vector vaccine containing genes for CEA, MUC-1, B7.1, ICAM-1, and LFA-3.

27. The kit of claim 26, further comprising at least one dose of a peripheral booster.

28. The kit of claim 26, further comprising at least one dose of rH-GM-CSF.

29. The kit of claim 26, further comprising instructions for the use thereof.

30. (canceled)

31. A method of decreasing the dose of a cancer immunotherapy vaccine comprising administering at least one intratumoral injection of tumor-antigen encoding poxvirus vaccine.

32. The method of claim 31, wherein the vaccine is a replication deficient recombinant fowlpox virus vector vaccine or a replication competent recombinant vaccinia virus vector.

33. (canceled)

34. (canceled)

35. (canceled)

36. (canceled)

37. (canceled)

38. The method of claim 31, wherein pancreatic cancer is treated.
